(19) United States
(12) Patent Application Publication
(10) Pub. No.: US 2023/0263716 A1
(43) Pub. Date: Aug. 24, 2023

(54) Compositions and methods for treating keratin materials

(71) Applicant: L’Oreal, Paris (FR)

(72) Inventors: Kazumitsu KAWAKAMI, Westfield, NJ (US); Ronak RUGHANI, Edison, NJ (US); Liliana XAVIER, Elizabeth, NJ (US); Aakash PAREKH, Edison, NJ (US)

(21) Appl. No.: 18/090,154

(22) Filed: Dec. 28, 2022

Related U.S. Application Data
(60) Provisional application No. 63/294,680, filed on Dec. 29, 2021.

(30) Foreign Application Priority Data
Feb. 21, 2022 (FR) 2201507

(51) Int. Cl. A61K 8/36; A61K 8/44; A61Q 5/02; A61Q 5/12

(52) U.S. Cl. CPC A61K 8/36; A61K 8/442; A61Q 5/02; A61Q 5/12; A61K 2800/40; A61K 2800/596

(57) ABSTRACT

The disclosure relates to compositions for treating keratin materials, comprising (a) at least one carboxylic acid or salt thereof, (b) panthenol, (c) at least one neutralizing agent, and (d) optionally at least one surfactant or salt thereof. Also disclosed are methods of treating, caring for, and/or strengthening keratin materials using the compositions.

### CROSS REFERENCE TO RELATED APPLICATIONS

This application claims priority to U.S. Provisional Application No. 63/294,680, filed Dec. 29, 2021, and FR 2201507 filed Feb. 21, 2022, both of which are incorporated herein in their entireties.

### TECHNICAL FIELD

The disclosure relates to compositions and methods for treating keratin materials, for example for cleansing, caring for, and/or strengthening keratin fibers.

### BACKGROUND

Consumers desire healthy hair, as healthy-looking hair is generally considered to be a sign of good health, good hygiene, and good hair-care practices. Thus, cleansing hair is routine. However, traditional hair cleansing compositions, while effective to cleanse the scalp or hair, may deleteriously affect the appearance and/or condition of the hair. For example, the scalp may appear and feel dry, and hair gloss and moisture balance may be negatively affected, making the hair more difficult to manage and style. Furthermore, dry hair that has been weakened or damaged is also prone to breakage and the formation of “split ends.”

Additionally, nutrition, environmental influences, physical and chemical treatments can also lead to dry hair damage that significantly weakens and dulls the hair over time. For instance, hair styles such as ponytails, buns, and braiding are quick and easy but when done too often and too tightly, can impart strain on the edges of the hair and cause a receding hair line. Hair also becomes physically damaged during detangling and styling. Excessive detangling can result in split ends and breakage.

Many chemical treatments are available for changing the appearance of hair. For example, hair may be lightened or bleached and oxidative dyes can be used to change the color of the hair. Chemical treatments for permanently straightening or curling the hair are also common. Chemical treatments are popular because their effects are long-lasting and can be drastic. Nonetheless, the biggest drawback to chemical treatments is the strain and damage they cause to the hair. This is because chemical treatments permanently change the chemical and physical structure of the hair. In addition, repeated us of heating appliances, such as flat irons and blow-dryers, to remove moisture from the surface of the hair cuticles during coloring or reshaping, may result in brittle, dry hair that become more vulnerable to breakage.

The environment also influences the health of hair. Regions with hard water can affect the look, feel and shine of the hair. This is because hard water leaves mineral deposits, which accumulate over time on the hair and eventually prevents moisture intake into the hair. The hair becomes dry, frizzy, and is prone to tangles. Environmental factors, such as strong sun, wind, cold air, extreme temperature variations and changes in air humidity can also damage the hair. The static and dry winter air contributes to moisture loss. Abrupt change from cold outdoor air to warm indoor air can cause the cuticle layers of the hair to lose moisture quickly into the atmosphere. Environmental effects on the hair, however, cannot be completely avoided.

As such, there remains a need for products that can impart strength to keratin fibers such as hair in order to combat damage. It has now been surprisingly and unexpectedly discovered that by treating keratin fibers such as hair with a combination of carboxylic acids, panthenol, and neutralizing agent, it is possible to impart internal fiber strength to the hair, while at the same time imparting overall healthy properties to the hair.

### SUMMARY

The disclosure relates to compositions and methods for treating, caring for, and/or strengthening keratin materials, e.g. keratin fibers such as hair.

In various embodiments, the disclosure relates to compositions for treating keratin materials, e.g. keratin fibers such as hair, comprising (a) at least about 0.1%, such as at least about 0.25%, at least about 0.5%, at least about 0.75%, or at least about 1% of at least one carboxylic acid or a salt thereof; (b) at least about 0.1%, such as at least about 0.25%, at least about 0.5%, at least about 0.75%, or at least about 1% of panthenol; (c) at least one neutralizing agent; and (d) optionally at least one surfactant chosen from amphoteric surfactants, anionic surfactants, cationic surfactants, nonionic surfactants, salts thereof, or mixtures thereof; wherein the composition has a pH ranging from about 3 to about 10, such as from about 4 to about 8, from about 5 to about 7, from about 5 to about 6.5, or from about 5.3 to about 6.3; and wherein all amounts are by weight, relative to the total weight of the composition. In various embodiments, the at least one carboxylic acid is chosen from oxalic acid, malonic acid, malic acid, glutaric acid, citraconic acid, citric acid, maleic acid, glycolic acid, succinic acid, adipic acid, tartaric acid, fumaric acid, sebacic acid, benzoic acid, salts thereof, or mixtures thereof. In various embodiments, the carboxylic acid is present in the composition in a total amount of at least about 0.5%, such as at least about 0.6%, at least about 0.7%, at least about 0.8%, at least about 0.9%, or at least about 1% by weight, relative to the total weight of the composition, and comprises citric acid. Preferably, the total amount of carboxylic acid(s) and panthenol are present in the composition so as to provide a weight ratio of carboxylic acid(s):panthenol ranging from about 1:5 to about 5:1, about 1:4 to about 4:1, or about 1:3 to about 3:1, such as ranging from about 1:2.5 to about 2.5:1, from about 1:2 to about 2:1, or from about 1:1.5 to about 1.5:1, or is about 1:1.

When present, compositions according to the disclosure may comprise at least one anionic surfactant chosen from acyl isethionates, acyl taurates, acyl sarcosinates, alkyl sulfonates, alkyl sulfosuccinates, alkyl ether sulfosuccinates, alkyl sulfoacetates, alkoxylated monoacids, salts thereof, or mixtures thereof. In some embodiments, the anionic surfactant comprises, consists essentially of, or consists of sodium cocoyl isethionate, sodium lauroyl isethionate, alkoxylated monoacids, or mixtures thereof. When present, the total amount of anionic surfactants ranges from about 1% to about 20%, such as from about 3% to about 18%, from about 5% to about 15%, or from about 8% to about 13% by weight, relative to the total weight of the composition.

When present, compositions according to the disclosure may comprise at least one amphoteric surfactant chosen from betaines, alkyl sultaines, alkyl amphoacetates, amphoproprionates, salts thereof, or mixtures thereof. In some embodiments, the amphoteric surfactant comprises, consists essentially of, or consists of betaines, for example (C₈-C₂₀) alkylbetaines, sulfobetaines, (C₈-C₂₀) alkylamido (C₆-C₈) alkylbetaines, (C₈-C₂₀) alkylamido (C₆-C₈) alkylsulfobetaines, salts thereof, or mixtures thereof. When present, the total amount of amphoteric surfactants ranges from about 0.1% to about 10%, such as from about 0.25% to about 8%, from about 0.5% to about 6%, or from about 1% to about 4% by weight, relative to the total weight of the composition.

When present, compositions according to the disclosure may comprise at least one nonionic surfactant chosen from alkyl polyglucosides, alkoxylated fatty alcohols, fatty amides, polyethoxylated fatty amides, polyglycerolated fatty amides, alkoxylated fatty acid esters of sorbitan comprising from 2 to 30 mol of ethylene oxide, polyoxyethylenated or non-polyoxyethylenated, saturated or unsaturated C₈-C₃₀ monoesters or polyesters of glycerol, polyoxyethylenated or non-polyoxyethylenated, saturated or unsaturated C₈-C₃₀ monoethers or polyethers of glycerol, fatty acid esters of sucrose, fatty acid esters of polyethylene glycol, polyethoxylated fatty acid mono- or diesters of glycerol, N-(C₆-C₂₄)alkylglucamine derivatives, polyglycerolated C₈-C₄₀ alcohols, ethylene glycol ethers of fatty alcohols, or mixtures thereof. In some embodiments, the nonionic surfactant comprises, consists essentially of, or consists of decylglucoside, laurylglucoside, cocoglucoside, caprylylglucoside, caprylyl/capryl glucoside, PEG-20 stearate, PEG-40 stearate, PEG-100 stearate, PEG-20 laurate, PEG-8 laurate, PEG-40 laurate, PEG-150 distearate, PEG-7 cocoate, PEG-9 cococate, PEG-8 oleate, PEG-10 oleate, PEG-40 hydrogenated castor oil, PPG-5-ceteth-20, or mixtures thereof. When present, the total amount of nonionic surfactants in the composition ranges from about 1% to about 20%, such as from about 5% to about 18%, from about 8% to about 16%, or from about 10% to about 15% by weight, relative to the total weight of the composition.

In various embodiments, compositions according to the disclosure may further comprise at least one conditioning compound chosen from cationic conditioning agents, silicone conditioning agents, non-silicone fatty compounds, or mixtures thereof.

Compositions according to the disclosure have a pH ranging from about 3 to about 10, such as from about 4 to about 10, from about 5 to about 9, from about 5 to about 8, from about 5 to about 7, from about 5 to about 6.5, or from about 5.3 to about 6.3.

In some embodiments, compositions according to the disclosure comprise (a) at least about 0.5%, such as at least about 0.75%, or at least about 1% of at least one carboxylic acid chosen from citric acid, maleic acid, malonic acid, salts thereof, or mixtures thereof, optionally including citric acid; (b) at least about 0.5%, such as at least about 0.75%, or at least about 1% of panthenol; (c) at least about 0.1%, such as at least about 0.25% of at least one neutralizing agent; and (d) at least one surfactant chosen from amphoteric surfactants, anionic surfactants, nonionic surfactants, salts thereof, or mixtures thereof; wherein the composition has a pH ranging from about 3 to about 10, such as from about 4 to about 8, from about 5 to about 7, about 5 to about 6.5, or from about 5.3 to about 6.3; and wherein all amounts are by weight, relative to the total weight of the composition. Preferably, the total amount of carboxylic acid(s) and panthenol are present in the composition so as to provide a weight ratio of carboxylic acid(s):panthenol ranging from about 1:5 to about 5:1, about 1:4 to about 4:1, or about 1:3 to about 3:1, such as ranging from about 1:2.5 to about 2.5:1, from about 1:2 to about 2:1, or from about 1:1.5 to about 1.5:1, or is about 1:1. The compositions optionally comprise at least one conditioning agent.

In further embodiments, compositions according to the disclosure comprise (a) at least about 0.75%, such as at least about 1% of citric acid and/or a salt thereof; (b) at least about 0.75%, such as at least about 1% of panthenol; (c) from about 0.1% to about 2%, such as from about 0.1% to about 1% of sodium hydroxide; and (d) at least one surfactant chosen from amphoteric surfactants, anionic surfactants, nonionic surfactants, salts thereof, or mixtures thereof; wherein the composition has a pH ranging from about 3 to about 10, such as from about 4 to about 9, from about 5 to about 8, from about 5 to about 7, from about 5 to about 6.5, or from about 5.3 to about 6.3; and wherein all amounts are by weight, relative to the total weight of the composition. Preferably, the total amount of carboxylic acid(s) and panthenol are present in the composition so as to provide a weight ratio of carboxylic acid(s):panthenol ranging from about 1:5 to about 5:1, about 1:4 to about 4:1, or about 1:3 to about 3:1, such as ranging from about 1:2.5 to about 2.5:1, from about 1:2 to about 2:1, or from about 1:1.5 to about 1.5:1, or is about 1:1. The compositions optionally comprise at least one conditioning agent.

In further embodiments, the disclosure relates to methods of cleansing keratin materials comprising applying compositions according to the disclosure to the keratin materials and optionally rinsing the keratin materials.

In still further embodiments, the disclosure relates to methods of strengthening keratin fibers, preferably hair, comprising applying compositions according to the disclosure to the keratin fibers, preferably hair, and optionally rinsing the keratin materials.

### DETAILED DESCRIPTION

The disclosure relates to, in various embodiments, compositions for treating keratin materials, preferably keratin fibers (e.g. hair), as well as to methods of using the compositions, for example methods for cleansing keratin materials and methods for imparting strength to or improving the strength of keratin fibers, as well as repairing and/or preventing damage of keratin fibers.

### I. Compositions

In exemplary and non-limiting embodiments, compositions according to the disclosure comprise (a) at least one carboxylic acid, salts thereof, or mixtures thereof; (b) panthenol; and (c) a neutralizer to maintain the pH of the composition in a range of from about 3 to about 10. In further embodiments, the compositions comprise (d) at least one surfactant chosen from amphoteric surfactants, anionic surfactants, cationic surfactants, nonionic surfactants, salts thereof, or mixtures thereof.

### Carboxylic Acid (s)

Compositions according to the disclosure comprise at least one carboxylic acid, salts thereof, or mixtures thereof. In various embodiments, the at least one carboxylic acid can be chosen from non-polymeric mono, di, or tri-carboxylic acids.

A non-polymeric mono, di, or tricarboxylic acid is an organic compound having one (mono), two (di), or three (tri) carboxylic acid groups (—COOH) and at least one carbon atom. The compounds typically have a molecular weight of less than about 500 g/mol, less than about 400 g/mol, or less than about 300 g/mol. Non-limiting examples of non-polymeric, mono, di, and/or tri-carboxylic acids include formic acid, acetic acid, propionic acid, butyric acid, valeric acid, caproic acid, entanthic acid, caprylic acid, pelargonic acid, capric acid, undecylic acid, lauric acid, tridecylic acid, lauric acid, tridecylic acid, myristic acid, pentadecylic acid, palmitic acid, margaric acid, stearic acid, nonadecylic acid, arachidic acid, lactic acid, oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, citric acid, isocitric acid, aconitric acid, propane-1,2,3-tricarboxylic acid, benzene-1,3,5-tricarboxylic acid, or salts of any of the foregoing.

In some embodiments, the compositions include one or more mono-carboxylic acids, salts thereof, or mixtures thereof. Non-limiting examples of mono-carboxylic acids include formic acid, acetic acid, propionic acid, butyric acid, valeric acid, caproic acid, entanthic acid, caprylic acid, pelargonic acid, capric acid, undecylic acid, lauric acid, tridecylic acid, lauric acid, tridecylic acid, myristic acid, pentadecylic acid, palmitic acid, margaric acid, stearic acid, nonadecylic acid, arachidic acid, and lactic acid.

In some cases, the compositions include one or more di-carboxylic acids, salts thereof, or mixtures thereof. Non-limiting examples of the di-carboxylic acids include oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, and 2,6-naphthalene dicarboxylic acid.

In some cases, the compositions may include one or more tricarboxylic acids, salts thereof, or mixtures thereof. Non-limiting examples of tricarboxylic acids include citric acid, isocitric acid, aconitric acid, propane-1,2,3-tricarboxylic acid, and benzene-1,3,5-tricarboxylic acid. In some instances, the compositions include citric acid and/or a salt thereof.

In various embodiments, the at least one carboxylic acid is chosen from oxalic acid, malonic acid, malic acid, glutaric acid, citraconic acid, citric acid, maleic acid, glycolic acid, succinic acid, adipic acid, tartaric acid, fumaric acid, sebacic acid, benzoic acid, salts thereof, or mixtures thereof. In at least some embodiments, the carboxylic acid comprises, consists essentially of, or consists of citric acid and/or a salt thereof.

In various embodiments, the total amount of carboxylic acids are in the compositions is at least about 0.1%, such as at least about 0.25%, at least about 0.5%, at least about 0.75%, or at least about 1% by weight, based on the total weight of the composition. For example, the total amount of carboxylic acids may be at least about 0.1%, at least about 0.2%, at least about 0.3%, at least about 0.4%, at least about 0.5%, at least about 0.6%, at least about 0.7%, at least about 0.8%, at least about 0.9%, or at least about 1% by weight, relative to the total weight of the composition. In other embodiments, the total amount of carboxylic acids may range from about 0.1% to about 5%, from about 0.2% to about 5%, from about 0.3% to about 5%, from about 0.4% to about 5%, from about 0.5% to about 5%, from about 0.6% to about 5%, from about 0.7% to about 5%, from about 0.8% to about 5%, from about 0.9% to about 5%, from about 1% to about 5%, from about 0.1% to about 4%, from about 0.2% to about 4%, from about 0.3% to about 4%, from about 0.4% to about 4%, from about 0.5% to about 4%, from about 0.6% to about 4%, from about 0.7% to about 4%, from about 0.8% to about 4%, from about 0.9% to about 4%, from about 1% to about 4%, from about 0.1% to about 3%, from about 0.2% to about 3%, from about 0.3% to about 3%, from about 0.4% to about 3%, from about 0.5% to about 3%, from about 0.6% to about 3%, from about 0.7% to about 3%, from about 0.8% to about 3%, from about 0.9% to about 3%, from about 1% to about 3%, from about 0.1% to about 2%, from about 0.2% to about 2%, from about 0.3% to about 2%, from about 0.4% to about 2%, from about 0.5% to about 2%, from about 0.6% to about 2%, from about 0.7% to about 2%, from about 0.8% to about 2%, from about 0.9% to about 2%, or from about 1% to about 2% by weight, relative to the total weight of the composition.

In still further embodiments, the amount of the at least one carboxylic acid may range from about 0.5% to about 15%, from about 0.5% to about 10%, from about 0.5% to about 5%, from about 0.5% to about 3%, from about 0.6% to about 15%, from about 0.6% to about 10%, from about 0.6% to about 5%, from about 0.6% to about 3%, from about 0.7% to about 15%, from about 0.7% to about 10%, from about 0.7% to about 5%, from about 0.7% to about 3%, from about 0.8% to about 15%, from about 0.8% to about 10%, from about 0.8% to about 5%, from about 0.8% to about 3%, from about 0.9% to about 15%, from about 0.9% to about 10%, from about 0.9% to about 5%, from about 0.9% to about 3%, from about 1% to about 15%, from about 1% to about 10%, from about 1% to about 5%, or from about 1% to about 3% by weight, relative to the total weight of the composition.

In some embodiments, the composition comprises citric acid and/or salts thereof in an amount of at least about 0.5%, at least about 0.6%, at least about 0.7%, at least about 0.8%, at least about 0.9%, or at least about 1% by weight, relative to the total weight of the composition. For example, in various embodiments, the composition comprises citric acid in an amount ranging from about 0.5% to about 5%, from about 0.5% to about 4%, from about 0.5% to about 3%, from about 0.5% to about 2%, from about 0.6% to about 5%, from about 0.6% to about 4%, from about 0.6% to about 3%, from about 0.6% to about 2%, from about 0.7% to about 5%, from about 0.7% to about 4%, from about 0.7% to about 3%, from about 0.7% to about 2%, from about 0.8% to about 5%, from about 0.8% to about 4%, from about 0.8% to about 3%, from about 0.8% to about 2%, from about 0.9% to about 5%, from about 0.9% to about 4%, from about 0.9% to about 3%, from about 0.9% to about 2%, from about 1% to about 5%, from about 1% to about 4%, from about 1% to about 3%, or from about 1% to about 2% by weight, relative to the total weight of the composition.

### Panthenol

The hair compositions include panthenol. Panthenol is an alcohol analog of pantothenic acid, having the following structure:

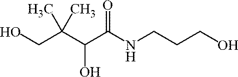

Panthenol is also called provitamin B5, which is converted to vitamin B5 only after it binds with other molecules. Panthenol can bind with the keratin fibers, even after the keratin fibers are washed or rinsed.

In some embodiments, the composition comprises panthenol in an amount of at least about 0.1%, such as at least about 0.25%, at least about 0.5%, at least about 0.75%, or at least about 1% by weight, based on the total weight of the composition. In further embodiments, the compositions comprise panthenol in an amount of at least about 0.5%, at least about 0.6%, at least about 0.7%, at least about 0.8%, at least about 0.9%, or at least about 1% by weight, relative to the total weight of the composition. For example, in various embodiments, the composition comprises panthenol in an amount ranging from about 0.5% to about 5%, from about 0.5% to about 4%, from about 0.5% to about 3%, from about 0.5% to about 2%, from about 0.6% to about 5%, from about 0.6% to about 4%, from about 0.6% to about 3%, from about 0.6% to about 2%, from about 0.7% to about 5%, from about 0.7% to about 4%, from about 0.7% to about 3%, from about 0.7% to about 2%, from about 0.8% to about 5%, from about 0.8% to about 4%, from about 0.8% to about 3%, from about 0.8% to about 2%, from about 0.9% to about 5%, from about 0.9% to about 4%, from about 0.9% to about 3%, from about 0.9% to about 2%, from about 1% to about 5%, from about 1% to about 4%, from about 1% to about 3%, or from about 1% to about 2% by weight, relative to the total weight of the composition.

In certain embodiments, it may be particularly advantageous to include total amounts of carboxylic acids and panthenol in compositions according to the disclosure in a certain weight ratio, which together can provide surprising and unexpected synergistic strengthening benefits to the hair. For example, in some embodiments, the total amount of carboxylic acids and panthenol are present in the composition in a weight ratio of carboxylic acid(s) to panthenol ranging from about 1:5 to about 5:1, such as about 1:4 to about 4:1 or about 1:3 to about 3:1, for example about 1:2.5 to about 2.5:1, about 1:2 to about 2:1, or about 1:1.5 to about 1.5:1, or about 1:1.

### Neutralizing Agent

The compositions according to the disclosure comprise at least one neutralizing agent. As used herein, the terms “neutralizing agent,” “neutralizer,” and variations thereof refer to a basifying agent used to neutralize the at least one carboxylic acid comprised in the compositions, in order to maintain the pH of the compositions in a range of about 3 to about 10.

In various embodiments, the total amount of neutralizers may vary, but typically is an amount necessary to maintain the pH of the composition in a range of about 3 to about 10, such as from about 4 to about 9, from about 5 to about 8, from about 5 to about 7, from about 5 to about 6.5, or from about 5.3 to about 6.3. For example, in some embodiments, the total amount of neutralizer may range from about 0.01% to about 5%, from about 0.01% to about 4%, from about 0.01% to about 3%, from about 0.01% to about 2%, from about 0.01% to about 1%, from about 0.05% to about 5%, from about 0.05% to about 4%, from about 0.05% to about 3%, from about 0.05% to about 2%, from about 0.05% to about 1%, from about 0.1% to about 5%, from about 0.1% to about 4%, from about 0.1% to about 3%, from about 0.1% to about 2%, from about 0.1% to about 1%, from about 0.5% to about 5%, from about 0.5% to about 4%, from about 0.5% to about 3%, from about 0.5% to about 2%, or from about 0.5% to about 1% by weight, relative to the total weight of the composition.

In some embodiments, the neutralizer comprises, consists essentially or, or consists of sodium hydroxide, and is present in an amount of at least about 0.1%, at least about 0.2%, at least about 0.3%, at least about 0.4%, or at least about 0.5% by weight, relative to the total weight of the composition, for example about 0.1% to about 1%, about 0.2% to about 1%, about 0.3% to about 1%, about 0.4% to about 1%, or about 0.5% to about 1%.

### Surfactants

In various embodiments, the hair treatment compositions comprise at least one surfactant chosen from amphoteric surfactants, anionic surfactants, cationic surfactants, nonionic surfactants, salts thereof, or mixtures thereof. In certain embodiments, the total amount of surfactants, regardless of the type(s) of surfactants, can vary. Nonetheless, when present, a typical amount of total surfactants typically ranges from about 0.1% to about 40% by weight, based on the total weight of the composition. For instance, in some cases, the total amount of surfactants ranges from about 0.1% to about 35%, about 0.1% to about 30 %, about 0.1% to about 25 %, about 0.1% to about 20%, about 0.5% to about 40%, about 0.5% to about 35%, about 0.5% to about 30%, about 0.5% to about 25%, about 0.5% to about 20%, about 1% to about 40%, about 1% to about 35%, about 1% to about 30%, about 1% to about 25%, about 1% to about 20%, about 5% to about 40%, about 5% to about 35%, about 5% to about 30%, about 5% to about 25%, about 5% to about 20%, about 10% to about 40%, about 10% to about 35%, about 10% to about 30%, about 10% to about 25%, about 10% to about 20%, about 15% to about 40%, about 15% to about 35%, about 15% to about 30%, about 15% to about 25%, about 15% to about 20%, about 20% to about 40%, about 20% to about 35%, about 20% to about 30%, about 25% to about 40%, about 25% to about 35%, or about 25% to about 30% by weight, based on the total weight of the composition.

### Anionic Surfactants

In various embodiments, the compositions according to the disclosure include one or more anionic surfactants. As used herein, the term “anionic surfactant” means a surfactant comprising, as ionic or ionizable groups, only anionic groups. These anionic groups may be chosen from the groups CO₂H, CO₂⁻, SO₃H, SO₃⁻, OSO₃H, OSO₃⁻ O₂PO₂H, O₂PO₂H, and O₂PO₂²⁻. Exemplary and non-limiting anionic surfactants include acyl isethionates, acyl taurates, acyl sarcosinates, alkyl sulfonates, alkyl sulfosuccinates, alkyl ether sulfosuccinates, alkyl sulfoacetates, alkoxylated monoacids, salts thereof, or mixtures thereof.

In various embodiments, the anionic surfactant may be chosen from sulfate surfactants, for example (C₆-C₂₄)alkyl sulfates, (C₆-C₂₄)alkyl ether sulfates, which are optionally ethoxylated, comprising from 2 to 50 ethylene oxide units, and mixtures thereof, in particular in the form of alkali metal salts or alkaline-earth metal salts, ammonium salts or amino alcohol salts. In some embodiments, the anionic surfactant may be chosen from (C₁₀-C₂₀)alkyl ether sulfates, and in particular sodium lauryl ether sulfate, optionally containing 2.2 mol of ethylene oxide. In other embodiments, sodium lauryl sulfate, sodium lauryl ether sulfate, ammonium lauryl sulfate, ammonium lauryl ether sulfate, sodium laureth sulfate, or mixtures thereof may be chosen.

In various exemplary embodiments, the anionic surfactant system comprises at least one anionic surfactant chosen from non-sulfate anionic surfactants, such as, for example, alkylsulfonates, alkylamide sulfonates, alkylarylsulfonates, alpha-olefin sulfonates, paraffin sulfonates, alkylsulfosuccinates, alkyl ether sulfosuccinates, alkylamide sulfosuccinates, alkyl sulfoacetates, acylsarcosinates, acylglutamates, alkylsulfosuccinamates, acylisethionates and N-acyltaurates, salts of alkyl monoesters and polyglycoside-polycarboxylic acids, acyllactylates, salts of D-galactoside uronic acids, salts of alkyl ether carboxylic acids, salts of alkyl aryl ether carboxylic acids, and salts of alkylamido ether carboxylic acids; or the non-salified forms of all of these compounds, the alkyl and acyl groups of all of these compounds containing from 6 to 24 carbon atoms and the aryl group denoting a phenyl group. Some of these compounds may be oxyethylenated and may comprise from 1 to 50 ethylene oxide units.

According to various embodiments, useful isethionate surfactants may be chosen from acyl isethionates of the following formulae (I) or (II):

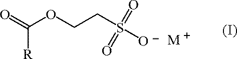

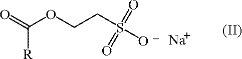

wherein R is chosen from hydrogen or an alkyl chain having from 1 to 30 carbon atoms, such as 6 to 24 carbon atoms, for example 8 to 20 carbon atoms, said chain being saturated or unsaturated, linear or branched, and M⁺ is a cation. Although sodium is shown as the cation in formula (II), it should be understood that the cation for both formula (I) and formula (II) may be any alkali metal ion such as sodium or potassium, ammonium ions, or alkanolammonium ions such as monoethanolammonium or triethanolammonium ions.

By way of non-limiting example, suitable acyl isethionate surfactants may include the reaction product of fatty acids esterified with isethionic acid and neutralized with sodium hydroxide. For example, acyl isethionates surfactants may be prepared by the reaction of an isethionate salt such as metal or ammonium isethionate and an a saturated or unsaturated, straight or branched, alkyl or alkenyl chain fatty acid having from 6 to 30 carbon atoms, such as from 8 to 22 carbon atoms, from 6 to 18 carbon atoms. Optionally, a mixture of aliphatic fatty acids may be used for the preparation of commercial fatty acyl isethionates surfactants. Suitable fatty acids for isethionate surfactants can be derived from coconut oil or palm kernel oil, for instance.

Non-limiting examples of acyl isethionate surfactants that may be used include sodium lauroyl isethionate, sodium lauroyl methyl isethionate, sodium oleoyl isethionate, sodium oleoyl methyl isethionate, sodium stearoyl isethionate, sodium stearoyl methyl isethionate, sodium myristoyl isethionate, sodium myristoyl methyl isethionate, sodium palmitoyl isethionate, sodium palmitoyl methyl isethionate, sodium cocoyl isethionate, sodium cocoyl methyl isethionate, a blend of stearic acid and sodium cocoyl isethionate, ammonium cocoyl isethionate, ammonium cocoyl methyl isethionate, and mixtures thereof. In some embodiments, the compositions comprise at least one anionic surfactant chosen from sodium cocoyl isethionate, sodium lauroyl isethionate, alkoxylated monoacids, or mixtures thereof.

Useful alkyl sulfonates include those of formula (III):

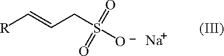

wherein R is selected from H or alkyl chain that has from 1 to 30 carbon atoms, such as from 6 to 24 carbon atoms, for example from 8 to 20 carbon atoms, said chain being saturated or unsaturated, linear or branched. It should be noted that although sodium is shown as the cation in the above formula (III), the cation may be any alkali metal ion such as sodium or potassium, ammonium ions, or alkanolammonium ions such as monoethanolammonium or triethanolammonium ions. In some instances, the alkyl sulfonate(s) are selected from C8-C16 alkyl benzene sulfonates, C10-C20 paraffin sulfonates, C10-C24 olefin sulfonates, salts thereof, and mixtures thereof.

By way of non-limiting example, alkyl sulfonates may be chosen from alkyl aryl sulfonates, primary alkane disulfonates, alkene sulfonates, hydroxyalkane sulfonates, alkyl glyceryl ether sulfonates, alpha-olefinsulfonates, sulfonates of alkylphenolpolyglycol ethers, alkylbenzenesulfonates, phenvlalkanesulfonates, alpha-olefinsulfonates, olefin sulfonates, alkene sulfonates, hydroxyalkanesulfonates and disulfonates, secondary alkanesulfonates, paraffin sulfonates, ester sulfonates, sulfonated fatty acid glycerol esters, and alpha-sulfo fatty acid methyl esters including methyl ester sulfonate.

Non-limiting examples of useful alkyl sulfosuccinates include those of formula (IV):

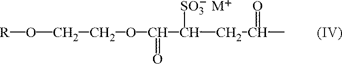

wherein R is a straight or branched chain alkyl or alkenyl group having from 10 to 22 carbon atoms, such as 10 to 20 carbon atoms, and M⁺ is a cation which can independently of each other be, for example, any alkali metal ion such as sodium, potassium, or ammonium, or alkanolammonium ions such as monoethanolammonium or triethanolammonium ions.

Non-limiting examples of alkyl sulfosuccinates salts include disodium oleamido MIPA sulfosuccinate, disodium oleamido MEA sulfosuccinate, disodium lauryl sulfosuccinate, disodium laureth sulfosuccinate, diammonium lauryl sulfosuccinate, diammonium laureth sulfosuccinate, dioctyl sodium sulfosuccinate, disodium oleamide MEA sulfosuccinate, sodium dialkyl sulfosuccinate, and mixtures thereof.

Exemplary and non-limiting alkyl sulfoacetates include C4-C18 fatty alcohol sulfoacetates and/or salts thereof, such as sodium lauryl sulfoacetate. Useful cations for the salts include any alkali metal ion such as sodium or potassium, ammonium ions, or alkanolammonium ions such as monoethanolammonium or triethanolammonium ions.

Non-limiting examples of alkoxylated monoacids include compounds corresponding to formula (V):

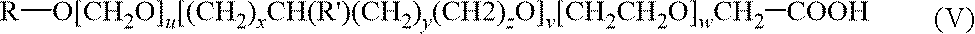

wherein:
R is a hydrocarbon radical containing from about 6 to about 40 carbon atoms;
u, v, and w, independently of one another, represent numbers of from 0 to 60;
x, y, and z, independently of one another, represent numbers of from 0 to 13;
R′ represents hydrogen or alkyl; and
the sum of x+y+z>0.

Compounds corresponding to formula (V) may be obtained by alkoxylation of alcohols R-OH with ethylene oxide as the sole alkoxide, or with several alkoxides and subsequent oxidation. The numbers u, v, and w each represent the degree of alkoxylation. Whereas, on a molecular level, the numbers u, v, and w and the total degree of alkoxylation can only be integers, including zero, on a macroscopic level they are mean values in the form of broken numbers.

In formula (V), R is linear or branched, acyclic or cyclic, saturated or unsaturated, aliphatic or aromatic, substituted or unsubstituted. R may be a linear or branched, acyclic C6-40 alkyl or alkenyl group, or a C1-40 alkyl phenyl group, for example a C8-22 alkyl or alkenyl group or a C4-18 alkyl phenyl group, such as a C12-18 alkyl group or alkenyl group or a C6-16 alkyl phenyl group; u, v, w, independently of one another, may be a number from 2 to 20, for example a number from 3 to 17, such as a number from 5 to 15; x, y, z, independently of one another, may be a number from 2 to 13, for example a number from 1 to 10, such as a number from 0 to 8.

By way of example only, useful alkoxylated monoacids include Butoxynol-5 Carboxylic Acid, Butoxynol-19 Carboxylic Acid, Capryleth-4 Carboxylic Acid, Capryleth-6 Carboxylic Acid, Capryleth-9 Carboxylic Acid, Ceteareth-25 Carboxylic Acid, Coceth-7 Carboxylic Acid, C9-11 Pareth-6 Carboxylic Acid, C11-15 Pareth-7 Carboxylic Acid, C12-13 Pareth-5 Carboxylic Acid, C12-13 Pareth-8 Carboxylic Acid, C12-13 Pareth-12 Carboxylic Acid, C12-15 Pareth-7 Carboxylic Acid, C12-15 Pareth-8 Carboxylic Acid, C14-15 Pareth-8 Carboxylic Acid, Deceth-7 Carboxylic Acid, Laureth-3 Carboxylic Acid, Laureth-4 Carboxylic Acid, Laureth-5 Carboxylic Acid, Laureth-6 Carboxylic Acid, Laureth-8 Carboxylic Acid, Laureth-10 Carboxylic Acid, Laureth-11 Carboxylic Acid, Laureth-12 Carboxylic Acid, Laureth-13 Carboxylic Acid, Laureth-14 Carboxylic Acid, Laureth-17 Carboxylic Acid, PPG-6-Laureth-6 Carboxylic Acid, PPG-8-Steareth-7 Carboxylic Acid, Myreth-3 Carboxylic Acid, Myreth-5 Carboxylic Acid, Nonoxynol-5 Carboxylic Acid, Nonoxynol-8 Carboxylic Acid, Nonoxynol-10 Carboxylic Acid, Octeth-3 Carboxylic Acid, Octoxynol-20 Carboxylic Acid, Oleth-3 Carboxylic Acid, Oleth-6 Carboxylic Acid, Oleth-10 Carboxylic Acid, PPG-3-Deceth-2 Carboxylic Acid, Capryleth-2 Carboxylic Acid, Ceteth-13 Carboxylic Acid, Deceth-2 Carboxylic Acid, Hexeth-4 Carboxylic Acid, Isosteareth-6 Carboxylic Acid, Isosteareth-11 Carboxylic Acid, Trudeceth-3 Carboxylic Acid, Trideceth-6 Carboxylic Acid, Trideceth-8 Carboxylic Acid, Trideceth-12 Carboxylic Acid, Trideceth-3 Carboxylic Acid, Trideceth-4 Carboxylic Acid, Trideceth-7 Carboxylic Acid, Trideceth-15 Carboxylic Acid, Trideceth-19 Carboxylic Acid, Undeceth-5 Carboxylic Acid and mixtures thereof. In some cases, ethoxylated acids include Oleth-10 Carboxylic Acid, Laureth-5 Carboxylic Acid, Laureth-11 Carboxylic Acid, and mixtures thereof.

Acyl amino acids that may be used include, but are not limited to, amino acid surfactants based on alanine, arginine, aspartic acid, glutamic acid, glycine, isoleucine, leucine, lysine, phenylalanine, serine, tyrosine, valine, sarcosine, threonine, and taurine. The most common cation associated with the acyl amino acid can be sodium or potassium. Alternatively, the cation can be an organic salt such as triethanolamine (TEA) or a metal salt. Non-limiting examples of useful acyl amino acids include those of formula (VI):

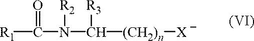

wherein R, R1, R2, and R3 are each independently selected from H or an alkyl chain having from 1 to 30 carbon atoms, said chain being saturated or unsaturated, linear or branched, and X is COO⁻ or SO₃⁻.

By way of example, useful acyl amino acids include acyl taurates, acyl glycinates, acyl glutamates, acyl sarcosinates, salts thereof, and mixtures thereof.

Exemplary useful acyl taurates include those of formula (VII):

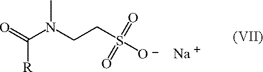

wherein R is selected from H or an alkyl chain having from 1 to 30 carbon atoms, such as from 6 to 24 carbon atoms, for example from 8 to 20 carbon atoms, said chain being saturated or unsaturated, linear or branched. It should be noted that although sodium is shown as the cation in the above formula (VII), the cation may be any alkali metal ion such as sodium or potassium, ammonium ions, or alkanolammonium ions such as monoethanolammonium or triethanolammonium ions. Non-limiting examples of acyl taurate salts include sodium cocoyl taurate, sodium methyl cocoyl taurate, and mixtures thereof.

Exemplary useful acyl glycinates include those of formula (VIII):

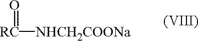

wherein R is an alkyl chain of 8 to 16 carbon atoms. It should be noted that although sodium is shown as the cation in the above formula (VIII), the cation may be any alkali metal ion such as sodium, potassium, or ammonium ions, or alkanolammonium ions such as monoethanolammonium or triethanolammonium ions. Non-limiting examples of acyl glycinates include sodium cocoyl glycinate, sodium lauroyl glycinate, sodium myristoyl glycinate, potassium lauroyl glycinate, and potassium cocoyl glycinate, and mixtures thereof.

Exemplary useful acyl glutamates include those of formula (IX):

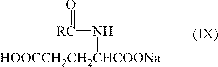

wherein R is an alkyl chain of 8 to 16 carbon atoms. It should be noted that although sodium is shown as the cation in the above formula (IX), the cation may be any alkali metal ion such as sodium, potassium, or ammonium ions, or alkanolammonium ions such as monoethanolammonium or triethanolammonium ions. Non-limiting examples of acyl glutamates include dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, sodium undecylenoyl glutamate, triethanolamine mono-cocoyl glutamate, triethanolamine lauroylglutamate, disodium cocoyl glutamate, and mixtures thereof.

Non-limiting examples of acyl sarcosinates include potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate, ammonium lauroyl sarcosinate, and mixtures thereof.

When the anionic surfactant(s) are in salt form, they may be chosen especially from alkali metal salts such as the sodium or potassium salt and preferably the sodium salt, ammonium salts, amine salts and in particular amino alcohol salts, or alkaline-earth metal salts such as the magnesium salt. Examples of amino alcohol salts that may especially be mentioned include monoethanolamine, diethanolamine and triethanolamine salts, monoisopropanolamine, diisopropanolamine or triisopropanol-amine salts, 2-amino-2-methyl-1-propanol salts, 2-amino-2-methyl-1,3-propanediol salts and tris(hydroxymethyl)aminomethane salts. Alkali metal or alkaline-earth metal salts and in particular the sodium or magnesium salts may be used. Exemplary salts of C₆-C₂₄ alkyl monoesters of polyglycoside-polycarboxylic acids include C₆-C₂₄ alkyl polyglycoside-citrates, C₆-C₂₄ alkyl polyglycoside-tartrates and C₆-C₂₄ alkyl polyglycoside-sulfo succinates.

In various embodiments, the total amount of anionic surfactants ranges from about 1% to about 20%, such as from about 3% to about 18%, from about 5% to about 15%, or from about 8% to about 13% by weight, relative to the total weight of the composition. For example, the total amount of anionic surfactant may range from about 1% to about 19%, from about 1% to about 18%, from about 1% to about 17%, from about 1% to about 16%, from about 1% to about 15%, from about 1% to about 14%, from about 1% to about 13%, from about 3% to about 20%, from about 3% to about 19%, from about 3% to about 17%, from about 3% to about 16%, from about 3% to about 15%, from about 3% to about 14%, from about 3% to about 13%, from about 5% to about 20%, from about 5% to about 19%, from about 5% to about 18%, from about 5% to about 17%, from about 5% to about 16%, from about 5% to about 14%, from about 5% to about 13%, from about 7% to about 20%, from about 7% to about 19%, from about 7% to about 18%, from about 7% to about 17%, from about 7% to about 16%, from about 7% to about 15%, from about 7% to about 14%, from about 7% to about 13%, from about 8% to about 20%, from about 8% to about 19%, from about 8% to about 18%, from about 8% to about 17%, from about 8% to about 16%, from about 8% to about 15%, from about 8% to about 14%, from about 10% to about 20%, from about 10% to about 19%, from about 10% to about 18%, from about 10% to about 17%, from about 10% to about 16%, from about 10% to about 15%, from about 10% to about 14%, or from about 10% to about 13% by weight, based on the total weight of the composition. In various non-limiting embodiments, the composition comprises at least one acyl isethionate, present in an amount ranging from about 5% to about 15%, about 5% to about 14%, about 5% to about 13%, about 5% to about 12%, about 6% to about 15%, about 6% to about 14%, about 6% to about 13%, about 6% to about 12%, about 7% to about 15%, about 7% to about 14%, about 7% to about 13%, about 7% to about 12%, about 8% to about 15%, about 8% to about 14%, about 8% to about 13%, about 8% to about 12%, about 9% to about 15%, about 9% to about 14%, about 9% to about 13%, about 9% to about 12%, about 10% to about 15%, about 10% to about 14%, about 10% to about 13%, or about 10% to about 12% by weight, based on the total weight of the composition.

### Cationic Surfactants

According to various embodiments, the compositions may comprise at least one cationic surfactant. However, in certain embodiments, the compositions are free or substantially free of cationic surfactants. Exemplary and non-limiting cationic surfactants include cationic amine-based or quaternary ammonium-based compounds, cationic cellulose-based compounds, cationic starch-based compounds, cationic galactomannan compounds, and cationic silicone compounds.

For example, cationic surfactants may be chosen from alkylpyridinium salts, ammonium salts of imidazoline, diquaternary ammonium salts, and ammonium salts containing at least one ester function. As a further example, cationic surfactants may be chosen from quaternary ammonium salts having the following formula (X):

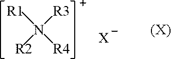

wherein R1 to R4, which may be identical or different, represent a linear or branched aliphatic radical containing from 1 to 30 carbon atoms, or an aromatic radical such as aryl or alkylaryl; the aliphatic radicals may optionally comprise heteroatoms (O, N, S or halogens) and may optionally be substituted, and X⁻ is an anion chosen from the group of halides, phosphates, acetates, lactates, C2-C6 alkyl sulfates and alkyl or alkylarylsulfonates. The aliphatic radicals are chosen, for example, from C12-C22 alkyl, alkoxy, C2-C6 polyoxyalkylene, alkylamide, (C12-C22)alkylamido(C2-C6)alkyl, (C12-C22)alkyl-acetate and hydroxyalkyl radicals, containing from 1 to 30 carbon atoms.

As a further example, quaternary ammonium salts containing at least one ester function, such as those of formula (XI) may be chosen:

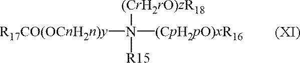

wherein:
R15 is chosen from C1-C6 alkyl radicals and C1-C6 hydroxyalkyl or dihydroxyalkyl radicals;
R16 is chosen from the radical R19-CO—, linear or branched, saturated or unsaturated C1-C22 hydrocarbon-based radicals R20, a hydrogen atom;
R18 is chosen from the radical R21-CO, linear or branched, saturated or unsaturated C1-C22 hydrocarbon-based radicals R22, a hydrogen atom;
R17, R19 and R21, which may be identical or different, are chosen from linear or branched, saturated or unsaturated C7-C21 hydrocarbon-based radicals;
r, n, and p, which may be identical or different, are integers ranging from 2 to 6;
y is an integer ranging from 1 to 10;
x and z, which may be identical or different, are integers ranging from 0 to 10; and
X⁻ is a simple or complex organic or mineral anion;
with the provisos that:
   the sum x+y+z is from 1 to 15,
   when x is 0, then R16 denotes R20, and
   when z is 0, then R18 denotes R22.

In formula (XI), the alkyl radicals R15 may be linear or branched, and more particularly linear. Preferably, R15 denotes a methyl, ethyl, hydroxyethyl or dihydroxypropyl radical, and more particularly a methyl or ethyl radical. Advantageously, the sum x+y+z is from 1 to 10. When R16 is a hydrocarbon-based radical R20, it may contain from 12 to 22 carbon atoms, or contain from 1 to 3 carbon atoms. When R18 is a hydrocarbon-based radical R22, it preferably contains 1 to 3 carbon atoms. Advantageously, R17, R19, and R21, which may be identical or different, are chosen from linear or branched, saturated or unsaturated C11-C21 hydrocarbon-based radicals, and more particularly from linear or branched, saturated or unsaturated C11-C21 alkyl and alkenyl radicals. Preferably, x and z, which may be identical or different, are equal to 0 or 1. Advantageously, y is equal to 1. Preferably, r, n and p, which may be identical or different, are equal to 2 or 3 and even more particularly equal to 2. The anion X⁻ is preferably a halide (chloride, bromide or iodide) or a C1-C4 alkyl sulfate, more particularly methyl sulfate. The anion X⁻ may also represent methanesulfonate, phosphate, nitrate, tosylate, an anion derived from an organic acid (such as acetate or lactate), or any other anion that is compatible with the ammonium containing an ester function.

The surfactants may be, for example, the salts (chloride or methyl sulfate) of diacyloxyethyldimethylammonium, of diacyloxyethylhydroxyethyldimethylammonium, of monoacyloxyethylhydroxyethyldimethylammonium, of triacyloxyethylmethylammonium, of monoacyloxyethylhydroxyethyldimethylammonium, and mixtures thereof. The acyl radicals preferably contain 14 to 18 carbon atoms and are more particularly derived from a plant oil, for instance palm oil or sunflower oil. When the compound contains several acyl radicals, these radicals may be identical or different.

Other suitable cationic surfactants are esterquats which are quaternary ammonium compounds having fatty acid chains containing ester linkages, such as, for example, dibehenoylethyl dimonium chloride, dipalmitoylethyl dimonium chloride, distearoylethyl dimonium chloride, ditallowoyl PG-dimonium chloride, dipalmitoylethyl hydroxyethylmonium methosulfate, distearoylethyl hydroxyethylmonium methosulfate, or mixtures thereof.

If present, the compositions comprise a total amount of cationic surfactants ranging from about 0.01% to about 2%, such as from about 0.01% to about 1.5%, from about 0.01% to about 1%, from about 0.01% to about 0.5%, from about 0.05% to about 2%, from about 0.05% to about 1.5%, from about 0.05% to about 1%, from about 0.05% to about 0.5%, from about 0.1% to about 2%, from about 0.1% to about 1.5%, from about 0.1% to about 1%, or from about 0.1% to about 0.5% by weight, relative to the total weight of the composition.

### Non-lonic Surfactants

Optionally, the composition may comprise one or more non-ionic surfactants. According to some embodiments, non-ionic surfactants may be chosen from saturated or unsaturated poly(oxyethylenated) C8-C30 monoesters or polyesters; saturated or unsaturated poly(oxyethylenated) C8-C30 alcohols; polyoxyethylenated or non-polyoxyethylenated, saturated or unsaturated C8-C30 monoesters or polyesters of sorbitan; polyoxyethylenated or non-polyoxyethylenated, saturated or unsaturated C8-C30 monoethers or polyethers of sorbitan; alkyl and polyalkyl glycosides or polyglycosides; saturated or unsaturated C8-C30 monoesters and polyesters of sucrose; polyoxyethylenated or non-polyoxyethylenated, saturated or unsaturated C8-C30 monoesters and polyesters of glycerol; polyoxyethylenated or non-polyoxyethylenated, saturated or unsaturated C8-C30 monoethers or polyethers of glycerol, or mixtures thereof.

As non-limiting examples of poly(oxyethylenated) saturated or unsaturated C8-C30 monoesters or polyesters, those with a number of ethylene oxide (EO) units ranging from 2 to 200, such as PEG-20 stearate, PEG-40 stearate, PEG-100 stearate, PEG-20 laurate, PEG-8 laurate, PEG-40 laurate, PEG-150 distearate, PEG-7 cocoate, PEG-9 cococate, PEG-8 oleate, PEG-10 oleate, PEG-40 hydrogenated castor oil, or mixtures thereof, may be chosen.

As saturated or unsaturated poly(oxyethylenated) C8-C30 alcohols, those with a number of ethylene oxide (EO) units ranging from 3 to 15, such as laureth-3, laureth-4, laureth-7, ceteth-5, ceteth-7, oleth-5, oleth-7, oleth-10, oleth-12, steareth-6, ceteareth-7, ceteareth-10, pareth-3, C12-15 pareth-3, C12-13 pareth-4, trideceth-3, trideceth-4, trideceth-5, trideceth-6, trideceth-7 and trideceth-10, or mixtures thereof, may be chosen.

Additionally, optionally polyoxyethylenated, saturated or unsaturated, C8-C30 monesters or polyesters of sorbitan, including those with a number of ethylene oxide (EO) units ranging from 0 to 100, such as sorbitan laurate, sorbitan laurate 4 EO, sorbitan laurate 20 EO (polysorbate 20), sorbitan palmitate 20 EO (polysorbate 40), sorbitan stearate 20 EO (polysorbate 60), sorbitan oleate 20 EO (polysorbate 80), sorbitan trioleate 20 EO (polysorbate 85), or mixtures thereof, may be used.

In some embodiments, optionally polyoxyethylenated, saturated or unsaturated, C8-C30 mono- or polyethers of sorbitan can include those with a number of ethylene oxide (EO) units ranging from 0 to 100.

Alkyl or polyalkyl glucosides or polyglucosides that may be used include those containing an alkyl group comprising from 6 to 30 carbon atoms and preferably from 6 to 18 or even from 8 to 16 carbon atoms, and containing a glucoside group preferably comprising from 1 to 5 and especially 1, 2, or 3 glucoside units. In certain embodiments, the composition comprises at least one alkyl polyglucoside having the following formula (XII):

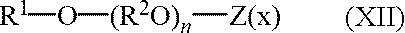

wherein:
R¹ is an alkyl group having from 8 to 18 carbon atoms;
R² is an ethylene or propylene group;
Z is a saccharide group with 5 to 6 carbon atoms;
n is an integer from 0 to 10; and
x is an integer from 1 to 5.

Nonlimiting examples of alkyl polyglucosides include lauryl glucoside, octyl glucoside, decyl glucoside, coco glucoside, caprylyl/capryl glucoside, and sodium lauryl glucose carboxylate. In certain embodiments, the at least one alkyl poly glucoside compound is selected from the group consisting of lauryl glucoside, decyl glucoside, coco glucoside, and mixtures thereof. In some embodiments, the hair treatment compositions comprise at least one alkyl polyglucoside, optionally including decyl glucoside.

Optionally polyoxyethylenated, saturated or unsaturated, mono- or polyesters of glycerol that may be chosen include those with a number of ethylene oxide (EO) units ranging from 0 to 100 and a number of glycerol units ranging from 1 to 30, for example, hexaglyceryl monolaurate, PEG-30 glyceryl stearate, polyglyceryl-2 laurate, polyglyceryl-10 laurate, polyglyceryl-10 stearate, polyglyceryl-10 oleate, PEG-7 glyceryl cocoate, PEG-20 glyceryl isostearate, or mixtures thereof.

Polyol esters may also be used. Non-limiting examples of polyol esters include propylene glycol dioctanoate, neopentyl glycol diheptanoate, diethylene glycol diisononanoate, neopentyl glycol dicaprate, neopentyl glycol dicaprylate/dicaprate, neopentyl glycol diethyl hexanoate, neopentyl glycol diheptanoate, neopentyl glycol diisononanoate, neopentyl glycol diisostearate, neopentyl glycol dilaurate, propanediol dicaprylate, propanediol diisostearate, propanediol dicaprylate/caprate, propanediol dibennzoate, propanediol didecanoate, propanediol dihexanoate, propylene glycol isostearate, propylene glycol caprylate, propylene glycol behenate, propylene glycol cocoate, propylene glycol laurate, propylene glycol linolenate, propylene glycol linoleate, propylene glycol myristate, propylene glycol oleate, propylene glycol stearate, or mixtures thereof. In some embodiments, polyol esters having alkoxylated groups such as PEG-55 propylene glycol oleate, PEG-6 propylene glycol caprylate/caprate, PEG-8 propylene glycol cocoate, PEG-25 propylene glycol stearate, PEG-120 propylene glycol stearate, or mixtures thereof may be chosen.

In some embodiments, ethylene glycol ethers of fatty alcohols may be used. Exemplary and non-limiting ethylene glycol ethers of fatty alcohols include the ceteth series of compounds such as ceteth-1 through ceteth-45, such as ceteth-7 through ceteth-20; the isoceteth series of compounds, for example isoceteth-20; the steareth series of compounds such as steareth-1 through steareth-100; the ceteareth series of compounds such as ceteareth 1 through ceteareth-50; the laureth series of compounds, such as laureth-7 through laureth-12; the pareth series of compounds, such as pareth-9 through pareth-15; propylene glycol ethers of the above such propylene glycol ethers of ceteth series of compounds including, for example, PPG-5-Ceteth-20; polyoxyethylene ethers or polyoxyethylene-polyoxypropylene ethers of branched alcohols, such branched alcohols including, for example, octyldodecyl alcohol, decyltetradecyl alcohol, dodecylpentadecyl alcohol, hexyldecyl alcohol, and isostearyl alcohol; polyoxyethylene-polyoxypropylene ethers of branched alcohols; or mixtures thereof.

In some embodiments, the compositions comprise at least one, for example at least two or at least three, nonionic surfactant(s) chosen from alkyl polyglucosides, alkoxylated fatty alcohols, fatty amides, polyethoxylated fatty amides, polyglycerolated fatty amides, alkoxylated fatty acid esters of sorbitan comprising from 2 to 30 mol of ethylene oxide, polyoxyethylenated or non-polyoxyethylenated, saturated or unsaturated C₈-C₃₀ monoesters or polyesters of glycerol, polyoxyethylenated or non-polyoxyethylenated, saturated or unsaturated C₈-C₃₀ monoethers or polyethers of glycerol, fatty acid esters of sucrose, fatty acid esters of polyethylene glycol, polyethoxylated fatty acid mono- or diesters of glycerol, N—(C₆-C₂₄)alkylglucamine derivatives, polyglycerolated C₈-C₄₀ alcohols, ethylene glycol ethers of fatty alcohols, or mixtures thereof. For example, the compositions may comprise at least one nonionic surfactant chosen from decylglucoside, laurylglucoside, cocoglucoside, caprylylglucoside, caprylyl/capryl glucoside, PEG-20 stearate, PEG-40 stearate, PEG-100 stearate, PEG-20 laurate, PEG-8 laurate, PEG-40 laurate, PEG-150 distearate, PEG-7 cocoate, PEG-9 cococate, PEG-8 oleate, PEG-10 oleate, PEG-40 hydrogenated castor oil, PPG-5-ceteth-20, or mixtures thereof.

The total amount of nonionic surfactants in the compositions may vary, but typically ranges from about 1% to about 20%, such as from about 5% to about 18%, from about 8% to about 16%, or from about 10% to about 15% by weight, including ranges and sub-ranges there between, relative to the total weight of the composition. For example, the total amount of nonionic surfactant may range from about 1% to about 19%, from about 1% to about 18%, from about 1% to about 17%, from about 1% to about 16%, from about 1% to about 15%, from about 1% to about 14%, from about 1% to about 13%, from about 3% to about 20%, from about 3% to about 19%, from about 3% to about 18%, from about 3% to about 17%, from about 3% to about 16%, from about 3% to about 15%, from about 3% to about 14%, from about 3% to about 13%, from about 5% to about 20%, from about 5% to about 19%, from about 5% to about 18%, from about 5% to about 17%, from about 5% to about 16%, from about 5% to about 15%, from about 5% to about 14%, from about 5% to about 13%, from about 7% to about 20%, from about 7% to about 19%, from about 7% to about 18%, from about 7% to about 17%, from about 7% to about 16%, from about 7% to about 15%, from about 7% to about 14%, from about 7% to about 13%, from about 8% to about 20%, from about 8% to about 19%, from about 8% to about 18%, from about 8% to about 17%, from about 8% to about 16%, from about 8% to about 15%, from about 8% to about 14%, from about 8% to about 13%, from about 10% to about 20%, from about 10% to about 19%, from about 10% to about 18%, from about 10% to about 17%, from about 10% to about 16%, from about 10% to about 15%, from about 10% to about 14%, or from about 10% to about 13% by weight, based on the total weight of the composition. In various non-limiting embodiments, the composition comprises at least one alkyl polyglucoside, and the total amount of alkyl polyglucosides is about 5% to about 15%, about 5% to about 14%, about 5% to about 13%, about 5% to about 12%, about 6% to about 15%, about 6% to about 14%, about 6% to about 13%, about 6% to about 12%, about 7% to about 15%, about 7% to about 14%, about 7% to about 13%, about 7% to about 12%, about 8% to about 15%, about 8% to about 14%, about 8% to about 13%, about 8% to about 12%, about 9% to about 15%, about 9% to about 14%, about 9% to about 13%, about 9% to about 12%, about 10% to about 15%, about 10% to about 14%, about 10% to about 13%, or about 10% to about 12% by weight, based on the total weight of the composition.

### Amphoteric Surfactants

Compositions according to the disclosure may optionally comprise at least one amphoteric surfactant. Non-limiting examples of amphoteric surfactants include betaines, sultaines, amphoacetates, amphoproprionates, salts thereof, and mixtures thereof. In various embodiments, the compositions may comprise at least one amphoteric surfactant chosen from betaines, alkyl sultaines, alkyl amphoacetates, amphoproprionates, salts thereof, or mixtures thereof.

In certain embodiments, betaines are chosen. In various embodiments, the compositions may comprise at least one amphoteric surfactant chosen from (C₈-C₂₀) alkylbetaines, sulfobetaines, (C₈-C₂₀) alkylamido (C₆-C₈) alkylbetaines, (C₈-C₂₀) alkylamido (C₆-C₈) alkylsulfobetaines, salts thereof, or mixtures thereof.

Betaines which can be used include those having the following formulae (XIII)-(XVI):

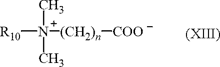

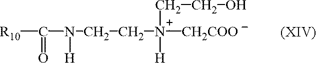

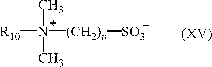

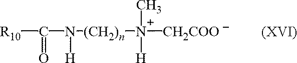

wherein in formulae (XIII)—(XVI)—
R10 is an alkyl group having from 6 to 20 carbon atoms; and
n is a integer from 1 to 3.

Non-limiting examples of betaines include coco betaine, cocoamidopropyl betaine, lauryl betaine, laurylhydroxy sulfobetaine, lauryldimethyl betaine, cocoamidopropyl hydroxysultaine, behenyl betaine, capryl/capramidopropyl betaine, lauryl hydroxysultaine, stearyl betaine, and mixtures thereof. In certain embodiments, the at least one betaine compound may be chosen from the group consisting of coco betaine, cocoamidopropyl betaine, behenyl betaine, capryl/capramidopropyl betaine, lauryl betaine, and mixtures thereof.

Hydroxyl sultaines useful in the compositions according to embodiments of the disclosure include the following formula (XVII):

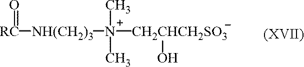

wherein R is an alkyl group having from 8 to 18 carbon atoms.

Useful alkylamphoacetates include those having the formula (XVIII):

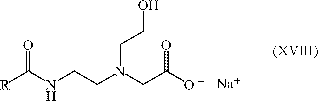

wherein R is an alkyl group having from 8 to 18 carbon atoms.

Useful alkyl amphodiacetates include those having the formula (XIX):

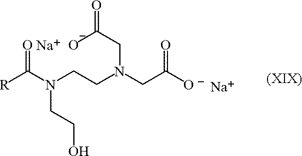

wherein R is an alkyl group having from 8 to 18 carbon atoms.

The amphoteric surfactants of the disclosure may be optionally quaternized secondary or tertiary aliphatic amine derivatives, in which the aliphatic group is a linear or branched chain comprising from 8 to 22 carbon atoms, said amine derivatives containing at least one anionic group, for instance a carboxylate, sulfonate, sulfate, phosphate or phosphonate group.

Among the optionally quaternized secondary or tertiary aliphatic amine derivatives that may be used, mention may also be made of the products of respective structures (A1) and (A2) below:

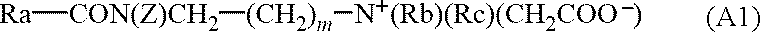

wherein:
Ra represents a C₁₀-C₃₀ alkyl or alkenyl group derived from an acid Ra—COOH preferably present in hydrolysed coconut oil, a heptyl group, a nonyl group or an undecyl group,
Rb represents a β-hydroxyethyl group,
Rc represents a carboxymethyl group;
m is equal to 0, 1 or 2, and
Z represents a hydrogen atom or a hydroxyethyl or carboxymethyl group;

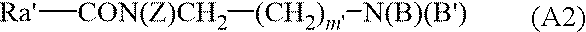

wherein:
B represents -CH₂CH₂OX′, with X′ representing -CH₂-COOH, CH₂-COOZ′, CH₂CH₂-COOH, -CH₂CH₂-COOZ′, or a hydrogen atom,
B′ represents -(CH₂)_{z}-Y’, with z ₌ 1 or 2, and Y’ representing COOH, COOZ’, CH₂-CHOH-SO₃H or -CH₂-CHOH-SO₃Z′,
m′ is equal to 0, 1 or 2,
Z represents a hydrogen atom or a hydroxyethyl or carboxymethyl group,
Z′ represents an ion resulting from an alkali or alkaline-earth metal, such as sodium, potassium or magnesium; an ammonium ion; or an ion resulting from an organic amine and in particular from an amino alcohol, such as monoethanolamine, diethanolamine and triethanolamine, monoisopropanolamine, diisopropanolamine or triisopropanolamine, 2-amino-2-methyl-1-propanol, 2-amino-2-methyl-1,3-propanediol and tris(hydroxymethyl)aminomethane, and
Ra′ represents a C₁₀-C₃₀ alkyl or alkenyl group of an acid Ra′COOH preferably present in hydrolysed linseed oil or coconut oil, an alkyl group, in particular a C₁₇ alkyl group, and its iso form, or an unsaturated C₁₇ group.

Exemplary amphoteric surfactants include sodium cocoamphoacetate, sodium lauroamphoacetate, sodium caproamphoacetate and sodium capryloamphoacetate. Further exemplary amphoteric surfactants include disodium cocoamphodiacetate, disodium lauroamphodiacetate, disodium caproamphodiacetate, disodium capryloamphodiacetate, disodium cocoamphodipropionate, disodium lauroamphodipropionate, disodium caproamphodipropionate, disodium capryloamphodi-propionate, lauroamphodipropionic acid and cocoamphodipropionic acid.

Non-limiting examples that may be mentioned include the cocoamphodiacetate sold by the company Rhodia under the trade name Miranol® C2M Concentrate, the sodium cocoamphoacetate sold under the trade name Miranol Ultra C 32, or the product sold by the company Chimex under the trade name CHIMEXANE HA.

Use may also be made of the compounds of formula (XX):

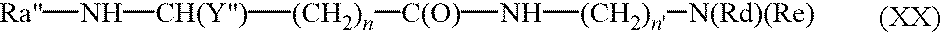

wherein:
Ra″ represents a C10-C30 alkyl or alkenyl group of an acid Ra″-C(O)OH preferably present in hydrolysed linseed oil or coconut oil;
Y″ represents the group -C(O)OH, -C(O)OZ″, -CH₂-CH(OH)-SO₃H or the group CH₂-CH(OH)-SO₃-Z″, with Z″ representing a cationic counterion resulting from an alkali metal or alkaline-earth metal, such as sodium, an ammonium ion or an ion resulting from an organic amine;
Rd and Re represent, independently of each other, a C₁-C₄ alkyl or hydroxyalkyl radical; and
n and n′ denote, independently of each other, an integer ranging from 1 to 3.

Exemplary compounds include sodium diethylaminopropylcoco-aspartamide, such as the one sold by the company Chimex under the name CHIMEXANE HB.

The total amount of amphoteric surfactants may range up to about 10%, such as from about 0.1% to about 10%, such as from about 0.25% to about 8%, from about 0.5% to about 6%, or from about 1% to about 4% by weight, relative to the total weight of the composition. For example, the amphoteric surfactants may be present in the composition in an amount ranging from about 0.1% to about 8%, about 0.1% to about 7%, about 0.1% to about 6%, about 0.1% to about 5%, about 0.1% to about 4%, about 0.1% to about 3%, about 0.25% to about 10%, about 0.25% to about 7%, about 0.25% to about 6%, about 0.25% to about 5%, about 0.25% to about 4%, about 0.25% to about 3%, about 0.5% to about 10%, about 0.5% to about 8%, about 0.5% to about 7%, about 0.5% to about 5%, about 0.5% to about 4%, about 0.5% to about 3%, about 0.75% to about 10%, about 0.75% to about 8%, about 0.75% to about 7%, about 0.75% to about 6%, about 0.75% to about 5%, about 0.75% to about 4%, about 0.75% to about 3%, about 1% to about 10%, about 1% to about 8%, about 1% to about 7%, about 1% to about 6%, about 1% to about 5%, about 1% to about 4%, about 1% to about 3%, about 1.25% to about 10%, about 1.25% to about 8%, about 1.25% to about 7%, about 1.25% to about 6%, about 1.25% to about 5%, about 1.25% to about 4%, about 1.25% to about 3%, about 1.5% to about 10%, about 1.5% to about 8%, about 1.5% to about 7%, about 1.5% to about 6%, about 1.5% to about 5%, about 1.5% to about 4%, or about 1.5% to about 3% by weight, relative to the total weight of the composition.

### Cosmetically Acceptable Solvent

The compositions according to the disclosure comprise at least one cosmetically acceptable solvent. In certain embodiments, the cosmetically acceptable solvents may be chosen from organic solvents, water-soluble solvents, water, or mixtures thereof.

The total amount of cosmetically acceptable solvent in the compositions may vary, but is typically from about 25% to about 95%, based on the total weight of the compositions. In some cases, the total amount of water is about 40% to about 90%, about 45% to about 85%, about 50% to about 75%, or about 55% to about 70%, including all ranges and sub-ranges there between, by weight based on the total weight of the composition.

### Conditioning Compounds

In various embodiments, it may be advantageous to include conditioning compounds in compositions according to the disclosure. In some embodiments, the compositions may comprise one or more cationic conditioning agents, silicone conditioning agents, non-silicone fatty compounds, or mixtures thereof.

In certain embodiments, compositions according to the disclosure comprise at least one cationic conditioning agent chosen from amidoamines, monoalkyl quaternary amines, dialkyl quaternary amines, polyquaternium compounds, or salts thereof.

Examples of amidoamines that are useful in the compositions of the instant disclosure include, but are not limited to the following: oleamidopropyl dimethylamine, stearamidopropyl dimethylamine, isostearamidopropyl dimethylamine, stearamidoethyl dimethylamine, lauramidopropyl dimethylamine, myristamidopropyl dimethylamine, behenamidopropyl dimethylamine, dilinoleamidopropyl dimethylamine, palmitamidopropyl dimethylamine, ricinoleamindopropyl dimethylamine, soyamidopropyl dimethylamine, wheat germamidopropyl dimethylamine, sunflowerseedamidopropyl dimethylamine, almondamidopropyl dimethylamine, avocadoamidopropyl dimethylamine, babassuamidopropyl dimethylamine, cocamidopropyl dimethylamine, minkamidopropyl dimethylamine, oatamidopropyl dimethylamine, sesamidopropyl dimethylamine, tallamidopropyl dimethylamine, brassicamidopropyl dimethylamine, olivamidopropyl dimethylamine, palmitamidopropyl dimethylamine, stearamidoethyldiethylamine, and mixtures thereof.

In further embodiments, cationic conditioning agents may be chosen from polyquaterium-10 (also called quaternized polyhydroxyethyl cellulose), cetrimonium chloride, behentrimonium chloride, behentrimonium methosulfate, steartrimonium chloride, stearalkonium chloride, dicetyldimonium chloride, hydroxypropyltrimonium chloride, cocotrimonium methosulfate, olealkonium chloride, steartrimonium chloride, babassuamidopropalkonium chloride, brassicamidopropyl dimethylamine, Quaternium-91, Salcare/PQ-37, Quaternium-22, Quaternium-87, lauryl betaine, Polyacrylate-1 Crosspolymer, steardimonium hydroxypropyl hydrolyzed wheat protein, behenamidopropyl PG-dimonium chloride, lauryldimonium hydroxypropyl hydrolyzed soy protein, Quaterium-8, or mixtures thereof.

In some instances, the cationic conditioning agents are cationic polymers. The term “cationic polymer” means any polymer comprising at least one cationic group and/or at least one group that may be ionized into a cationic group.

In various embodiments, cationic polymers that may be chosen include, but are not limited to: polyquaternium 4, polyquaternium 6, polyquaternium 7, polyquaternium 10, polyquaternium 11, polyquaternium 16, polyquaternium 22, polyquaternium 28, polyquaternium 32, polyquaternium-46, polyquaternium-51, polyquaternium-52, polyquaternium-53, polyquaternium-54, polyquaternium-55, polyquaternium-56, polyquaternium-57, polyquaternium-58, polyquaternium-59, polyquaternium-60, polyquaternium-63, polyquaternium-64, polyquaternium-65, polyquaternium-66, polyquaternium-67, polyquaternium-70, polyquaternium-73, polyquaternium-74, polyquaternium-75, polyquaternium-76, polyquaternium-77, polyquaternium-78, polyquaternium-79, polyquaternium-80, polyquaternium-81, polyquaternium-82, polyquaternium-84, polyquaternium-85, polyquaternium-86, polyquaternium-87, polyquaternium-90, polyquaternium-91, polyquaternium-92, polyquaternium-94, guar hydroxypropyltrimonium chloride, or mixtures thereof.

Useful and non-limiting silicone conditioning agents that can be included in compositions according to the disclosure include, but are not limited to, polyorganosiloxanes, polyalkylsiloxanes, polyarylsiloxanes, polyalkarylsiloxanes, polyestersiloxanes, and a mixture thereof. For example, dimethicone, cyclomethicone (cyclopentasiloxane), amodimethicone, trimethyl silyl amodimethicone, phenyl trimethicone, trimethyl siloxy silicate, polymethylsilsesquioxane, or mixtures thereof may be chosen.

In some embodiments, the compositions according to the disclosure are free or substantially free of silicones. For example, the compositions may in some embodiments include less than about 3%, less than about 2%, or less than about 1% of silicones. In other embodiments, the compositions comprise one or more silicones, for example in an amount of from about 0.01% up to about 5%, such as from about 0.01% to about 4%, from about 0.01% from about 0.01% to about 3%, from about 0.01% to about 2%, or from about 0.01% to about 1% by weight, relative to the total weight of the composition.

Useful and non-limiting examples of non-silicone fatty compounds include natural oils, such as coconut oil; hydrocarbons, such as mineral oil and hydrogenated polyisobutene; fatty alcohols, such as octyldodecanol; esters, such as C12-C15 alkyl benzoate; diesters, such as propylene dipelarganate; and triesters, such as glyceryl trioctanoate. Examples of useful low viscosity oils include isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, coco-dicaprylate/caprate, decyl isostearate, isodecyl oleate, isodecyl neopentanoate, isohexyl neopentanoate, octyl palmitate, dioctyl malate, tridecyl octanoate, myristyl myristate, octododecanol, or combinations of octyldodecanol, acetylated lanolin alcohol, cetyl acetate, isododecanol, polyglyceryl-3-diisostearate, or mixtures thereof. Examples of useful high viscosity oils include castor oil, lanolin and lanolin derivatives, triisocetyl citrate, sorbitan sesquioleate, C10-C18 triglycerides, caprylic/capric/triglycerides, coconut oil, corn oil, cottonseed oil, glyceryl triacetyl hydroxystearate, glyceryl triacetyl ricinoleate, glyceryl trioctanoate, hydrogenated castor oil, linseed oil, mink oil, olive oil, palm oil, illipe butter, rapeseed oil, soybean oil, sunflower seed oil, tallow, tricaprin, trihydroxystearin, triisostearin, trilaurin, trilinolein, trimyristin, triolein, tripalmitin, tristearin, walnut oil, wheat germ oil, cholesterol, or mixtures thereof.

### Additives

The compositions according to the disclosure may also further comprise additives chosen from nacreous agents, dyes or pigments, fragrances, mineral, plant or synthetic oils, waxes, thickening agents/rheology modifiers, plant extracts, vitamins other than panthenol (e.g. vitamin E, vitamin C, etc.), amino acids, electrolytes (e.g. sodium chloride), opacifying agents, proteins including ceramides, UV-screening agents, antidandruff agents, hair-loss counteractants, hair restorers, preservatives, or mixtures thereof. A person skilled in the art will take care to select the optional additives and the amount thereof such that they do not harm the properties of the compositions of the disclosure.

In various embodiments, the additives are generally present in an amount ranging up to about 20% by weight of active material relative to the total weight of the composition, such as up to about 15%, up to about 10%, up to about 5%, up to about 3%, or up to about 2%, such as from about 0.01% to about 5% or about 0.1% to about 3%.

The compositions may be transparent or semi-transparent, and their viscosities may vary but may be often similar to or greater than that of conventional cleansing, shampooing, conditioning, and/or other hair care compositions. Accordingly, in some instances, the viscosity of a composition disclosed herein may range from about 3000 to about 10,000 cPs, for example from about 3500 to about 8500 cPs, or from about 4000 to about 7000 cPs when measured at 25° C. using a Brookfield Viscometer DV-II+ Pro, RV-3 disc at 20 rpm.

The compositions may have a pH ranging from about 3 to about 10, such as from about 4 to about 10, from about 5 to about 9, from about 5 to about 8, from about 5 to about 7, from about 5 to about 6.5, or from about 5.3 to about 6.3.

In various embodiments, the compositions according to the disclosure are stable, meaning that no phase separation or significant change in pH or viscosity is seen when stored at a temperature ranging from about 4° C. to about 45° C., such as from about 10° C. to about 37° C., or from about 20° C. to about 30° C., for at least about 4 weeks, at least about 6 weeks, or at least about 8 weeks.

In various embodiments, the compositions according to the disclosure may be in the form of shampoos, conditioners, hair gels, hair creams, hair lotions, hair sprays, etc.

It has surprisingly and unexpectedly been discovered that compositions according to the disclosure may provide improved strength to keratin fibers that have been treated with the compositions. The compositions may, in various embodiments, be a rinse-off shampoo, which can be used alone or in combination with one or more other compositions such as a conditioner, and/or may be a leave-in hair care composition. The compositions may also be a leave-in conditioning shampoo or rinse-off conditioning shampoo. In addition to provide strengthening effect to hair when applied to hair and scalp, the compositions may impart to hair treated thereby one or more properties such as smoothness, conditioning, excellent detangling, anti-frizz, ease of shaping and/or combing, anti-static, clean and/or smooth appearance, with no weigh-down.

### II. Methods

Methods according to the disclosure relate to methods of cleansing keratin materials, e.g. keratin fibers such as hair, using compositions according to the disclosure. Further methods relate to treating, caring for, and/or conditioning keratin fibers, in particular, for strengthening keratin fibers and minimizing or repairing damage to keratin fibers, using the compositions described herein.

The methods generally comprise applying a sufficient amount of a composition disclosed herein on the keratin fibers, optionally leaving the composition on for a leave-on time, and optionally, subsequently rinsing the composition therefrom.

In some embodiments, the methods of the disclosure include a single treatment or multiple treatments repeatedly using a composition according to the disclosure over a period of time. For instance, the treatments of the methods may be repeated daily, every-other-day, three or four times per week, once or twice per week, one, two, or three times per month, etc. The repeated treatments may be carried out for a period of time, for example, for one week, two weeks, one month, three months, six months, or longer. As some of the hair-treatment compositions in the methods may be shampoos and/or conditioners, the methods may be carried out according to an individual’s normal shampooing (and optional conditioning) routine.

According to some embodiments, an effective amount of a hair-treatment composition (including, for example, shampoos and/or conditioners) is applied to the hair, for example, hair that has been wetted with water. The hair-treatment compositions may also be applied to dry hair. Furthermore, the hair-treatment compositions may be applied to hair that has been artificially dyed, pigmented, or chemically permed or straightened, or hair that has not been artificially dyed, pigmented, or chemically permed or straightened.

In some embodiments, where a hair-treatment compositions according to the disclosure is a rinse-off composition, the methods may include applying a sufficient amount of the composition on the hair and allowing the composition to remain on the hair for any desired amount of time (leave-on time), for example from about a few seconds (e.g. about 5, 10, 20, or 30 seconds) to about 1, 2, 3, 5, 10, 20, or 30 minutes, or longer. After the leave-on time has expired, the hair-treatment composition may be rinsed from the hair, usually with water.

In various embodiments, where the composition is a leave-in product, the methods may include applying a sufficient amount of the leave-in product to hair (either wet, damp, or dry hair), and optionally, drying and/or styling the hair. The composition may be left on the hair for any period of time, such as a few hours or a few days, or until the next washing or rinsing of the hair. In addition, before applying the compositions to the hair, the composition may optionally be lathered before application to the keratin materials, e.g. in the hands, or may be lathered while on the keratin materials.

The methods of treating and/or caring for the hair and/or scalp according to the disclosure may, in various embodiments, impart internal fiber strength to the hair, even after the composition is rinsed off. In addition, hair treated with the compositions according to the disclosure may have greater ease of detangling, greater smoothness, discipline without a greasy coating or weighed-down feeling, moisturized feel, split-end seal, and/or reduced static, may be sleeker, and/or may have greater frizz control, relative to hair not having been treated with a composition according to the disclosure.

Having described the many embodiments of the present invention in detail, it will be apparent that modifications and variations are possible without departing from the scope of the invention defined in the appended claims. Furthermore, it should be appreciated that all examples in the disclosure, while illustrating many embodiments of the invention, are provided as non-limiting examples and are, therefore, not to be taken as limiting the various aspects so illustrated. It is to be understood that all definitions herein are provided for the present disclosure only.

As used herein, the terms “comprising,” “having,” and “including” (or “comprise,” “have,” and “include”) are used in their open, non-limiting sense.

In this application, the use of the singular includes the plural unless specifically stated otherwise. The singular forms “a,” “an,” “the,” and “at least one” are understood to encompass the plural as well as the singular unless the context clearly dictates otherwise. The expression “one or more” and “at least one” are interchangeable and expressly include individual components as well as mixtures/combinations. Likewise, the term “a salt thereof” also relates to “salts thereof.” Thus, where the disclosure refers to “at least one element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two or more of A, B, C, D, E, F, one or more salts of A, one or more salts of B, one or more salts of C, one or more salts of D, one or more salts of E, and one or more salts of F may be included.

The term “and/or” should be understood to include both the conjunctive and the disjunctive. For example, “water and/or non-aqueous solvents” means “water and non-aqueous solvents” as well as “water or non-aqueous solvents,” and expressly covers instances of either.

As used herein, the phrases “and mixtures thereof,” “and a mixture thereof,” “and combinations thereof,” “and a combination thereof,” “or mixtures thereof,” “or a mixture thereof,” “or combinations thereof,” and “or a combination thereof,” are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

For purposes of the disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

All ranges and amounts given herein are intended to include sub-ranges and amounts using any disclosed point as an end point, and all endpoints are intended to be included unless expressly stated otherwise. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not expressly stated, unless expressly stated otherwise. Similarly, a range given of “about 1% to 10%” is intended to have the term “about” modifying both the 1% and the 10% endpoints. The term “about” is used herein to indicate a difference of up to +/- 10% from the stated number, such as +/- 9%, +/- 8%, +/- 7%, +/-6%, +/- 5%, +/- 4%, +/- 3%, +/- 2%, or +/- 1%. Likewise, all endpoints of ranges are understood to be individually disclosed, such that, for example, a range of 1:2 to 2:1 is understood to disclose a ratio of both 1:2 and 2:1.

As used herein, if a component is described as being present “in an amount up to” a certain amount, it is intended that such component is, in fact, present in the composition, i.e. is present in an amount greater than 0%.

All amounts and ratios herein are given based upon the total weight of the composition, unless otherwise indicated. Unless otherwise indicated, all percentages herein are by weight of active material.

As used herein, the phrase “applying a composition onto keratin fibers” and variations thereof are intended to mean contacting the keratin fibers such as hair with at least one of the compositions of the disclosure, in any manner. It may also mean contacting the keratin fibers with an effective amount of the composition.

As used herein, the term “salts” referred to throughout the disclosure may include salts having a counterion such as an alkali metal, alkaline earth metal, or ammonium counterion. This list of counterions, however, is non-limiting. Salts also include a dissociated form of a compound, e.g. in an aqueous solution.

As used herein, the term “substantially free” or “essentially free” means the specific material may be present in small amounts that do not materially affect the basic and novel characteristics of the compositions according to the disclosure. For instance, there may be less than 2% by weight of a specific material added to a composition, based on the total weight of the compositions (provided that an amount of less than 2% by weight does not materially affect the basic and novel characteristics of the compositions according to the disclosure. Similarly, the compositions may include less than 2%, less than 1.5%, less than 1%, less than 0.5%, less than 0.1%, less than 0.05%, or less than 0.01%, or none of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The term “substantially free” or “essentially free” as used herein may also mean that the specific material is not added to the composition but may still be present in a raw material that is included in the composition.

As used herein, the terms “treat,” “treated,” “treatment,” and variations thereof is not intended to be limiting, but rather is merely intended to indicate that one or more compositions is applied to the hair, and optionally removed from the hair. For example, hair that is “treated” with a composition according to the disclosure may have had the composition applied, and/or may have had the composition applied and removed, e.g. by rinsing or towel drying. As a further example, hair that is “treated” with a composition according to the disclosure may have had the composition applied, and/or may have had the composition applied and rinsed from the hair.

“Keratin fibers” include, but are not limited to, human hair. In an embodiment, the terms “keratin fibers,” “hair,” and the like refer in particular to hair on the scalp, and exclude eyelashes.

A “hair-treatment composition” is a composition that is applied to the hair to achieve a particular effect. Non-limiting examples of hair-treatment compositions include shampoos, conditioners, hair gels, hair sprays, hair rinses, hair lotions, rinse-out hair masques, etc. Hair-treatment compositions are often rinsed from the hair after a period of time (“leave-in” or “leave-on” time), e.g., after the hair-treatment composition has had sufficient time to impart the desired cosmetic effect to the hair. The components for application to the hair may be applied simultaneously in a single hair-treatment composition or can be applied separately in a sequence (as part of a “bundle treatment” or “sequential treatment”).

As used herein, the terms “carboxylic acids” and “surfactants” include salts thereof, even if not explicitly stated.

As used herein, the term “conditioning” means imparting to hair fibers at least one property chosen from combability, moisture-retentivity, luster, shine, and softness. The state of conditioning can be evaluated by any means known in the art, such as, for example, measuring, and comparing, the ease of combability of the treated hair and of the untreated hair in terms of combing work, and consumer perception.

As used herein, “cosmetic composition” encompasses many types of compositions for application to keratin fibers such as hair, for example, hair lotions, hair creams, hair gel creams, hair conditioners, hair masques (masks), hair sprats, etc., which can be used either as leave-in or rinse-off treatments or products.

A “rinse-off” product refers to a composition such as a hair-treatment composition that is rinsed and/or washed with water either after or during the application of the composition onto the keratin fiber, and before drying and/or styling said keratin fiber. At least a portion, and typically most, of the composition is removed from the keratin fibers during the rinsing and/or washing.

A “leave-in” or “leave-on” product refers to a composition such as a hair-treatment composition that is not rinsed and/or washed with water or acceptable solvent after the application of the composition onto the keratin fiber; instead, the composition is allowed to remain on the fibers for a period of time, such as, for example, for at least 1 hour, 2 hours, 3 hours, 4 hours, up to 8 hours, or overnight.

“Polymers,” as defined herein, include homopolymers as well as copolymers formed from at least two different types of monomers.

“Hydrocarbons,” as used herein, include alkanes, alkenes, and alkynes, wherein the alkanes comprise at least one carbon, and the alkenes and alkynes each comprise at least two carbons; further wherein the hydrocarbons may be chosen from linear hydrocarbons, branched hydrocarbons, and cyclic hydrocarbons; further wherein the hydrocarbons may optionally be substituted; and further wherein the hydrocarbons may optionally further comprise at least one heteroatom intercalated in the hydrocarbon chain.

“Silicone compound,” as used herein, includes, for example, silica, silanes, silazanes, siloxanes, and organosiloxanes; and refers to a compound comprising at least one silicon; wherein the silicone compound may be chosen from linear silicone compounds, branched silicone compounds, and cyclic silicone compounds; further wherein the silicone compound may optionally be substituted; and further wherein the silicone compound may optionally further comprise at least one heteroatom intercalated in the silicone chain, wherein the at least one heteroatom is different from the at least one silicon.

“Substituted,” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalkyl groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

Unless otherwise expressly stated, it is in no way intended that any method set forth herein be construed as requiring that its steps be performed in a specific order. Accordingly, where a method does not expressly recite that a particular order of steps must be followed or it is not otherwise specifically stated that the steps are to be limited to a specific order, it is no way intended that any particular order be inferred.

It should also be understood that the precise numerical values used in the specification and claims form additional embodiments of the disclosure, and are intended to include any ranges which may be narrowed to any two end points disclosed within the exemplary ranges and values provided, as well as the specific end points themselves.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The examples that follow serve to illustrate embodiments of the disclosure without, however, being limiting in nature.

### EXAMPLES

The following examples serve to illustrate the embodiments of the disclosure without however exhibiting a limiting character. The Examples are intended to be non-restrictive and explanatory only, with the scope of the invention being defined by the claims. In these examples the amounts of the composition ingredients are given as weight percentages of active ingredients relative to the total weight of the composition.

### Example 1 - Shampoo Compositions

Shampoo compositions 1A, 1B, and 1C according to the disclosure, with the formulations set forth in Table 1, were prepared.

TABLE 1

Inventive Shampoo Compositions

| | | | |
|---|---|---|---|
| | 1A | 1B | 1C |
| ACRYLATES/BEHENETH-25 METHACRYLATE COPOLYMER | 0.075 | 0.075 | 0.075 |
| CETRIMONIUM CHLORIDE | 0.003 | 0.003 | 0.003 |
| SODIUM HYDROXIDE | 0.5 | 0.5 | 0.5 |
| SODIUM PHYTATE | 0.15 | | |
| SODIUM BENZOATE | 0.5 | 0.5 | 0.5 |
| SODIUM COCOYL ISETHIONATE | 11 | 11 | 11 |
| COCAMIDOPROPYL BETAINE | 2.6 | 2.6 | 2.6 |
| DIMETHICONE | 0.75 | 0.75 | 0.75 |
| PEG-55 PROPYLENE GLYCOL OLEATE | 0.4 | 0.6 | 0.4 |
| COCO-BETAINE | 0.1 | 0.1 | 0.1 |
| DECYL GLUCOSIDE | 11.7 | 11.7 | 11.7 |
| AMODIMETHICONE | 0.2 | 0.2 | 0.2 |
| POLYQUATERNIUM-67 | 0.2 | 0.2 | 0.2 |
| SODIUM CHLORIDE | 0.5 | 0.5 | 0.5 |
| CITRIC ACID | 1.0 | 1.0 | 1.0 |
| TRIDECETH-6 | 0.02 | 0.02 | 0.02 |
| PANTHENOL | 1.0 | 1.0 | 1.0 |
| CARBOMER | 0.3 | 0.3 | 0.3 |
| PEG-150 DISTEARATE | 0.5 | 0.5 | 0.5 |
| PPG-5-CETETH-20 | 0.5 | 0.5 | 0.5 |
| GLYCOL DISTEARATE | 0.12 | 0.12 | 0.12 |
| COCO-CAPRYLATE/CAPRATE | 0.2 | 0.2 | 0.2 |
| ADDITIVES (fragrance, preservatives, thickeners, plant extracts, amino acids/proteins, emulsifiers, pH adjusters) | <1 | <2 | <1 |
| SOLVENT (water + non-aqueous solvents) | QS | QS | QS |

Each of compositions 1A, 1B, and 1C was in liquid form and stable, having a pH ranging from about 5-6 and a viscosity ranging from about 4000-7000 cPs when measured at 25° C. using a Brookfield Viscometer DV-II+ Pro, RV-3 disc at 20 rpm.

### Example 2 - Comparative Shampoo Composition

Comparative shampoo composition C1, with the formulation set forth in Table 2, was prepared.

TABLE 2

| | Comparative Shampoo Composition |
|---|---|
| | C1 |
| HEXYLENE GLYCOL | 1.25 |
| SODIUM LAURETH SULFATE | 8.4 |
| SODIUM BENZOATE | 0.5 |
| GLYCERIN | 2.0 |
| PROPYLENE GLYCOL | 0.43 |
| COCAMIDE MEA | 0.5 |
| COCO-BETAINE | 3.0 |
| LAURETH-12 | 0.25 |
| SALICYLIC ACID | 0.20 |
| SODIUM CHLORIDE | 2.15 |
| ADDITIVES (plant extracts, dyes/piqments, etc.) | <1 |
| WATER | QS |
| VISCOSITY (Brookfield Viscometer DV-II+ Pro, RV-3 disc at 20 rpm, 25° C.) | 4000 cPs |

### Example 3 - Cyclic Fatigue Tensile Testing

Cyclic fatigue tensile testing was performed to assess the hair strengthening benefits of compositions according to the disclosure.

Inventive shampoo compositions 1A and 1C (Table 1), a commercially available conditioner 3A, a commercially available leave-in treatment composition 3B, and comparative shampoo composition C1 (Table 2) were used in this testing. The ingredients of compositions 3A and 3B are listed in Table 3, as follows.

TABLE 3

| Commercial Conditioner | Commercial Leave-in Treatment Composition/Lotion |
|---|---|
| 3A | 3B |
| WATER, CETEARYL ALCOHOL, GLYCERIN, BEHENTRIMONIUM CHLORIDE, DIMETHICONE, BISCETEARYL AMODIMETHICONE, CETYL ESTERS, CITRIC ACID, ISOPROPYL ALCOHOL, DICETYL-DIMONIUM CHLORIDE, PARFUM/FRAGRANCE, SODIUM CITRATE, PHENOXYETHANOL, CANDELILLA CERA/ CANDELILLA WAX, ISOPROPYL MYRISTATE, HYDROXYPROPYL GUAR, LIMONENE, COCO-BETAINE, AMODIMETHICONE, CETRIMONIUM CHLORIDE, DILAURYL HIODIPROPIONATE, TRIDECETH-10, SODIUM CHLORIDE, LINALOOL, PEG-100 STEARATE, CITRONELLOL, STEARETH-6, TRIDECETH-3, ACETIC ACID, TRISODIUM HEDTA | WATER, DIMETHICONE, CETEARYL ALCOHOL, AMODIMETHICONE, PHENOXYETHANOL, POLYQUATERNIUM-37, DIMETHICONOL, PARFUM/FRAGRANCE, PROPYLENE GLYCOL DICAPRYLATE/DICAPRATE, TRIDECETH-5, STEARETH-20, CETYL HYDROXYETHYL-CELLULOSE, GLYCERIN, CITRIC ACID, LIMONENE, PPG-1 TRIDECETH-6, TRIDECETH-10, SODIUM HYDROXIDE, CHLORHEXIDINE DIGLUCONATE, SORBITAN OLEATE, ACETIC ACID, LINALOOL, CITRONELLOL, CITRAL, EUGENOL |

This testing was carried out on bleached Caucasian hair swatches having about 50 hair fibers, at about 20° C. and 45% relative humidity, with the specific compositions and applications thereof as shown in Table 4, below.

After the hair was treated, the cycles to break (CTB) was evaluated using a Cyclic Fatigue Tensile Tester (CFTT). A higher number of CTB indicates a more durable (stronger) hair fiber. Obtained CTB scores were analyzed with the Kaplan Meier statistical test. The average CTB for each treatment group, presented in median value, is shown in Table 4, below.

TABLE 4

| Treatment Group | Compositions / Applications | Average CTB | Variation | Count |
|---|---|---|---|---|
| K | C1 × 1 | 2270 | 3.859e+06 | 49 |
| L | (1A + 3A) × 6 | 3721 | 1.931e+07 | 48 |
| M | (1A + 3A + 3B) × 6 | 2889 | 6.197e+07 | 49 |
| A | C1 × 1 | 2051 | 9.190e+07 | 50 |
| F | 1C × 6 | 2512 | 6.356e+07 | 46 |

As shown in Table 4, the inventive compositions, alone or in a bundle, provide significantly improved strength to the hair. The differences shown in Table 4 are both surprising and statistically significant.

These results demonstrate that hair treated with shampoos according to the present disclosure surprisingly exhibited unexpected significantly improved strength, compared to the hair not so treated.

The above examples demonstrate that the synergistic combination of carboxylic acid, panthenol, and neutralizing agent provides unexpectedly superior strengthening properties to hair, and the compositions provide excellent cleansing and conditioning properties to the hair.

1-67. (canceled)

68. A composition for treating keratin fibers comprising:
(a) at least about 0.5% of at least one carboxylic acid;
(b) at least about 0.1% of panthenol;
(c) at least one neutralizing agent; and
(d) at least one surfactant chosen from amphoteric surfactants, anionic surfactants, cationic surfactants, nonionic surfactants, salts thereof, or mixtures thereof;
wherein the composition has a pH ranging from about 3 to about 10; and
wherein all amounts are by weight, relative to the total weight of the composition.

69. The composition according to claim 68, wherein the (a) at least one carboxylic acid is chosen from oxalic acid, malonic acid, malic acid, glutaric acid, citraconic acid, citric acid, maleic acid, glycolic acid, succinic acid, adipic acid, tartaric acid, fumaric acid, sebacic acid, benzoic acid, salts thereof, or mixtures thereof.

70. The composition according to claim 68, comprising (d) at least one anionic surfactant chosen from acyl isethionates, acyl taurates, acyl sarcosinates, alkyl sulfonates, alkyl sulfosuccinates, alkyl ether sulfosuccinates, alkyl sulfoacetates, alkoxylated monoacids, salts thereof, or mixtures thereof.

71. The composition according to claim 68, comprising (d) at least one anionic surfactant, wherein the total amount of anionic surfactants ranges from about 1% to about 20% by weight, relative to the total weight of the composition.

72. The composition according to claim 68, comprising (d) at least one amphoteric surfactant chosen from betaines, alkyl sultaines, alkyl amphoacetates, amphoproprionates, salts thereof, or mixtures thereof.

73. The composition according to claim 68, comprising (d) at least one amphoteric surfactant, wherein the total amount of amphoteric surfactants ranges from about 0.1% to about 10% by weight, relative to the total weight of the composition.

74. The composition according to claim 68, comprising (d) at least one nonionic surfactant chosen from decylglucoside, laurylglucoside, cocoglucoside, caprylylglucoside, caprylyl/capryl glucoside, PEG-20 stearate, PEG-40 stearate, PEG-100 stearate, PEG-20 laurate, PEG-8 laurate, PEG-40 laurate, PEG-150 distearate, PEG-7 cocoate, PEG-9 cococate, PEG-8 oleate, PEG-10 oleate, PEG-40 hydrogenated castor oil, PPG-5-ceteth-20, or mixtures thereof.

75. The composition according to claim 68, comprising (d) at least one nonionic surfactant, wherein the total amount of nonionic surfactants ranges from about 1% to about 20% by weight, relative to the total weight of the composition.

76. The composition according to claim 68, further comprising at least one conditioning compound chosen from cationic conditioning agents, silicone conditioning agents, non-silicone fatty compounds, or mixtures thereof.

77. The composition according to claim 68, further comprising at least one electrolyte.

78. The composition according to claim 68, wherein the at least one neutralizing agent comprises sodium hydroxide, and is present in an amount sufficient to maintain the pH of the composition in the range of about 5 to about 7.

79. The composition according to claim 68, wherein the total amounts of (a) carboxylic acid(s) and (b) panthenol are present in the composition such that the weight ratio of carboxylic acid(s):panthenol ranges from about 1:3 to about 3:1.

80. A composition for treating keratin fibers comprising:
(a) at least one carboxylic acid chosen from citric acid, maleic acid, malonic acid, salts thereof, or mixtures thereof, where the total amount of citric acid, maleic acid, malonic acid, and salts thereof is at least about 0.5%;
(b) panthenol, where the total amount of panthenol is at least about 0.5%;
(c) at least one neutralizing agent, where the total amount of neutralizing agents is at least about 0.1%; and
(d) at least one surfactant chosen from amphoteric surfactants, anionic surfactants, nonionic surfactants, salts thereof, or mixtures thereof;
wherein the composition has a pH ranging from about 3 to about 10; and
wherein all amounts are by weight, relative to the total weight of the composition.

81. The composition according to claim 80, wherein the at least one carboxylic acid comprises citric acid and/or a salt thereof.

82. The composition according to claim 80, further comprising at least one conditioning compound chosen from cationic conditioning agents, silicone conditioning agents, non-silicone fatty compounds, or mixtures thereof.

83. The composition according to claim 80, further comprising at least one electrolyte.

84. The composition according to claim 80, wherein the at least one neutralizing agent comprises sodium hydroxide, and is present in an amount sufficient to maintain the pH of the composition in the range of about 5 to about 7.

85. The composition according to claim 80, wherein the total amounts of (a) carboxylic acid(s) and (b) panthenol are present in the composition such that the weight ratio of carboxylic acid(s):panthenol ranges from about 1:3 to about 3:1.

86. A method for treating keratin fibers comprising:
(1) applying to the keratin fibers a composition comprising:
(a) at least about 0.5% of at least one carboxylic acid;
(b) at least about 0.1% of panthenol;
(c) at least one neutralizing agent; and
(d) at least one surfactant chosen from amphoteric surfactants, anionic surfactants, cationic surfactants, nonionic surfactants, salts thereof, or mixtures thereof;
wherein the composition has a pH ranging from about 3 to about 10; and
wherein all amounts are by weight, relative to the total weight of the composition; and
(2) optionally rinsing the keratin fibers.

87. The method according to claim 86, wherein the total amounts of (a) carboxylic acid(s) and (b) panthenol are present in the composition such that the weight ratio of carboxylic acid(s):panthenol ranges from about 1:3 to about 3:1.
